(19) United States
(12) Patent Application Publication
(10) Pub. No.: US 2019/0255333 A1
(43) Pub. Date: Aug. 22, 2019

(54) SYSTEMS AND METHODS FOR CURRENT STIMULATION UTILIZING ACTIVE CHARGE BALANCE PHASE

(71) Applicant: BIOTRONIK SE & CO. KG, Berlin (DE)

(72) Inventors: MARCELO BARU, TUALATIN, OR (US); BRAD MCMILLAN, LAKE OSWEGO, OR (US); ASHOK NEDUNGADI, LAKE OSWEGO, OR (US)

(21) Appl. No.: 16/253,914

(22) Filed: Jan. 22, 2019

Related U.S. Application Data
(60) Provisional application No. 62/631,976, filed on Feb. 19, 2018.
(60) Provisional application No. 62/770,858, filed on Nov. 23, 2018.

(51) Int. Cl. A61N 1/36; A61N 1/05

(52) U.S. Cl. CPC A61N 1/36157; A61N 1/0551; A61N 1/36125; A61N 1/36153

(57) ABSTRACT

A medical device for electrical stimulation includes at least two electrodes and electric circuitry for performing active charge compensation. The electrodes are connected to the electric circuitry and the electric circuitry is configured to perform the active charge compensation using a passive element or an active element. A method for controlling a medical device and a medical device system are also disclosed.

### CROSS-REFERENCE TO RELATED APPLICATION

This application claims the priority, under 35 U.S.C. § 119(e), of U.S. Provisional Patent Application Nos. 62/631,976 filed Feb. 19, 2018 and 62/770,858 filed Nov. 23, 2018; the prior applications are herewith incorporated by reference in their entirety.

### BACKGROUND OF THE INVENTION

### Field of the Invention

The present invention relates to neurostimulation applications with charge balancing/charge compensation capability. In particular, the present invention refers to multi-electrode stimulation applications.

Electrical neurostimulation applications, in particular spinal cord stimulation (SCS), are demanding implantable pulse generator (IPG) architectures that can simultaneously source and sink currents from multiple electrodes, inject large charges, support high pulsing rates (thus active charge balancing), with reduced electrode areas (to improve selectivity) and without therapy interruption.

Unlike cardiac pacemakers, fractal coating is not typically utilized in neurostimulation. SCS for example uses Pt/Ir electrodes which present a small electrode-tissue double-layer capacitance (in the pF range). Those small capacitances, combined with the in series DC blocking capacitors utilized for protection, may result in substantial voltage accumulation following a stimulation phase. During an active charge balance phase, that accumulated voltage will reverse polarity and appear at the driving electronics front-end, which needs to handle it properly in order not to trigger parasitic diodes and/or protection elements (e.g. for electrostatic discharge ESD) which otherwise would cause uncontrolled unbalanced stimulation and establish hazardous electrode potentials at steady-state.

U.S. Pat. No. 9,757,565 discloses adjusting the compliance voltage (i.e. total voltage overhead in that Invention Disclosure) for the current sources and/or sinks in an implantable stimulator device, during a stimulation pulse, by monitoring the voltage drop across the elements that implement such currents and maintaining them near optimal values.

U.S. Pat. No. 8,538,548 discloses calculating the minimum overhead voltage required for the stimulating currents based on impedance measurements and minimum compliance voltages required to guarantee saturation of the transistors implementing the current sources/sinks for efficient stimulation.

U.S. Patent No. 2016/0367813 discloses circuitry for dealing with “capacitive-looking” electrodes (e.g. capacitive behavior of tissue) in bipolar stimulation using an H-bridge which can be extended to more than one stimulus electrode when they shared a return electrode. In particular, U.S. Publication No. 2016/0367813 states that many bidirectional neural interface applications (e.g. closed-loop deep-brain stimulation) may require digital signal processing within the implant, so as to control the stimulator in response to recorded neural activity. To minimize size and complexity of the implant, they propose using a single bulk-CMOS chip to have analog and digital functionality on the same chip which imposes a severe limitation on the stimulator voltage compliance. To deal with large voltages that result from driving charge-balanced, biphasic stimulus current through the electrode-tissue interface impedance, for capacitive-looking electrodes, they propose utilizing the accumulated voltage after the leading stimulus pulse is delivered to start delivering the balancing stimulus current and detect when the electrode-tissue has sufficiently discharged to connect a supply voltage to deliver the remaining balancing stimulus current.

U.S. Provisional Application No. 62/631,976 describes systems and methods for simultaneous multi-electrode stimulation, providing solutions to the accumulated voltage reverse polarity during active charge balancing for a simultaneous multi-electrode, multi-current therapy. The content of that former application is hereby entirely incorporated by reference.

### SUMMARY OF THE INVENTION

Active biphasic stimulation is formed of a stimulation phase, an interphase period where electrodes electrically float (i.e. are not driven by the IPG), an active charge balance phase, and a passive charge balance phase where participating electrodes are short-circuited (equivalent to autoshort in pacemakers) to prevent voltage runaway caused by charge mismatches in the biphasic stimulation.

It is accordingly an object of the invention to provide systems and methods for current stimulation utilizing active charge balance phase, which overcome the hereinafore-mentioned disadvantages of the heretofore-known systems and methods of this general type, based on an accumulated-voltage compensation during the active charge balance phase, with compensation either using a passive element or an active element.

In a preferred embodiment, using passive-element compensation, the accumulated voltage accumulated during the stimulation phase is compensated via an ohmic voltage drop. A programmable resistor in series with the balance path, either connected to V_{IStim} (overhead voltage required for the stimulation phase) or to system ground V_{SS}, depending on the stimulation configuration (three examples will be discussed below), cancels out the effect of the accumulated voltage of the stimulation phase. In an alternative passive-element compensation embodiment, the accumulated voltage during the stimulation phase is compensated via a capacitive voltage drop.

In a preferred active-element compensation embodiment, the accumulated voltage during the stimulation phase is compensated via an active element, preferably a voltage follower, having an output which provides the total voltage overhead for the active balance. The output voltage may be determined on a pulse-by-pulse basis, during the interphase period, by sampling the maximum accumulated voltage that occurred during the stimulation phase and subtracting it from the total overhead voltage utilized during the stimulation phase to drive the active balance phase.

To implement either passive-element embodiment, in a first approach, the accumulated voltage during the stimulation phase is determined. To do so, in a stage pre-therapy, the maximum accumulated voltage in any of the equivalent capacitances in the stimulation paths is measured using a unipolar stimulation phase as programmed and a measurement at the end of the programmed interphase period to account for voltage changes that occur during the latter as chemical reactions (triggered by the stimulation phase) keep occurring. At the end of the measurement, passive charge balance is applied so that therapy can start as programmed. A quick binary search permits determining the value of the resistor or capacitor that will compensate the accumulated charge during the active charge balance phase.

In an alternative approach, an estimation of the programmable resistor to be used during the active charge balance phase (e.g. during therapy amplitude ramp up) is rather performed assuming the total capacitance in series with the stimulation path is constant and considering the programmed stimulation phase maximum current. During the start of the associated active charge balance phase, the programmable resistor is quickly adjusted to an optimum value to compensate the accumulated voltage with the ohmic voltage drop. In this approach there is no pre-therapy stage, and the value of the programmable resistor is estimated and determined during the active charge balance phase associated with the stimulation phase.

According to an embodiment of the invention, a medical system for electrical stimulation is proposed, comprising:
at least two electrodes,
electric circuitry for managing the accumulated voltage generated by a stimulation phase during a reverse active charge balancing phase to prevent additional currents through tissue other than the programmed one, wherein the electrodes are connected to the circuitry, and
wherein the electric circuitry is configured to manage such accumulated-voltage using either a passive element or an active element.

According to an embodiment of the invention, the accumulated voltage is compensated via an ohmic voltage drop in series with the active charge-balancing path.

According to an embodiment of the invention, the accumulated voltage is compensated via a capacitive voltage drop in series with the active charge-balancing path.

According to an embodiment of the invention, the electric circuitry includes a voltage follower, wherein the voltage follower is configured to be used for accumulated voltage compensation.

According to an embodiment of the invention, the voltage follower is configured such that its output adjusts the total voltage overhead needed for the active charge-balancing path.

According to an embodiment of the invention, the electric circuitry includes a programmable resistor, wherein the programmable resistor is configured to be used for accumulated-voltage compensation.

According to an embodiment of the invention, the medical system is configured to automatically determine the resistance required for accumulated-voltage compensation prior to delivering therapy, wherein the estimated resistance is then programmed to the programmable resistor.

According to an embodiment of the invention, the medical system is configured to estimate the resistance required for accumulated-voltage compensation, wherein the estimated resistance is then programmed to the programmable resistor.

According to an embodiment of the invention, the medical system is configured to adjust the resistance required for accumulated-voltage compensation, wherein the resistance is programmed to the programmable resistor, as it delivers therapy.

Moreover, according to an embodiment of the invention, a method for controlling a medical system having at least two electrodes is proposed, comprising the steps of:
delivering electrical stimulation via the electrodes, and
managing accumulated voltage generated by a stimulation phase during a reverse active charge balancing phase to prevent additional currents through tissue other than the programmed one.

According to an embodiment of the inventive method, the accumulated voltage is compensated via an ohmic voltage drop or a capacitive voltage drop in series with the active charge-balancing path.

According to an embodiment of the inventive method, the method further comprises the steps of:
performing a voltage measurement in the electrical paths associated with the stimulation electrodes prior to therapy delivery, and/or
performing a voltage measurement in the electrical paths associated with the stimulation electrodes at the end of an interphase period.

According to an embodiment of the inventive method, the voltage measurement includes measuring the accumulated voltage of capacitances in the electrical paths for electrical stimulation.

According to an embodiment of the inventive method, the method further includes the steps of:
determining the impedance required for accumulated-voltage compensation, and
programming the determined impedance to the programmable resistor.

According to an embodiment of the inventive method, the method further includes the steps of:
estimation of an impedance required for accumulated-voltage compensation, and
programming the estimated impedance to the programmable resistor.

According to an embodiment of the invention, the medical system includes an implantable medical device for neurostimulation.

During therapy settling until steady-state is reached (i.e biphasic stimulation charge mismatch equals the charge balanced by passive balance), therapy amplitude ramp up, and during therapy delivery with long duty cycle on or continuously where the electrode-tissue-electrode complex impedances may vary (e.g. caused by patient body posture changes), both the stimulation and active charge balance phases are adjusted on-the-fly. The stimulation phase will be adjusted as proposed in U.S. Provisional Application No. 62/631,976 and U.S. Provisional Application No. 62/631,976 whereas for the active charge balance phase measurements at the beginning of it will permit assessing whether the ASIC pads are within a certain voltage window band and adjust compensation accordingly if outside.

Other features which are considered as characteristic for the invention are set forth in the appended claims.

Although the invention is illustrated and described herein as embodied in systems and methods for current stimulation utilizing active charge balance phase, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a schematic representation of a classical multi-electrode system;

FIG. 2 is a schematic representation of a front-end circuit of an IPG;

FIG. 3 is a schematic representation of a High Matching circuit with Ohmic Voltage Drop Compensation, injection of stimulation phase;

FIG. 4 is a schematic representation of a High Matching circuit with Ohmic Voltage Drop Compensation, in which a measurement phase takes place after the programmed interphase period;

FIG. 5 is a schematic representation of an ASIC, where injection of the balance current through the programmable resistor block alone and binary searches for the value that approximates the measured V_{Acc} takes place;

FIG. 6 is a schematic representation of a High Matching configuration with Capacitive Voltage Drop Compensation;

FIG. 7 is a schematic representation of a charging of capacitor similar to FIG. 5;

FIG. 8 is a schematic representation of a circuit for removing accumulated extra charge;

FIG. 9 is a schematic representation of a configuration with denominated Normal Matching with Ohmic Drop Voltage Compensation;

FIG. 10 is a schematic representation of a preferred active-element compensation embodiment;

FIG. 11 is a schematic representation of an implementation of a circuit for handling the current mismatch between the sum of sinking currents and the sum of sourcing currents without using an auxiliary electrode; and

FIG. 12 is a schematic representation of an embodiment of stimulation and active charge balance phases for the Current Steering configuration.

### DETAILED DESCRIPTION OF THE INVENTION

Before describing each embodiment for different possible stimulation configurations, some technical background will be described to better understand the voltage accumulation during the stimulation phase.

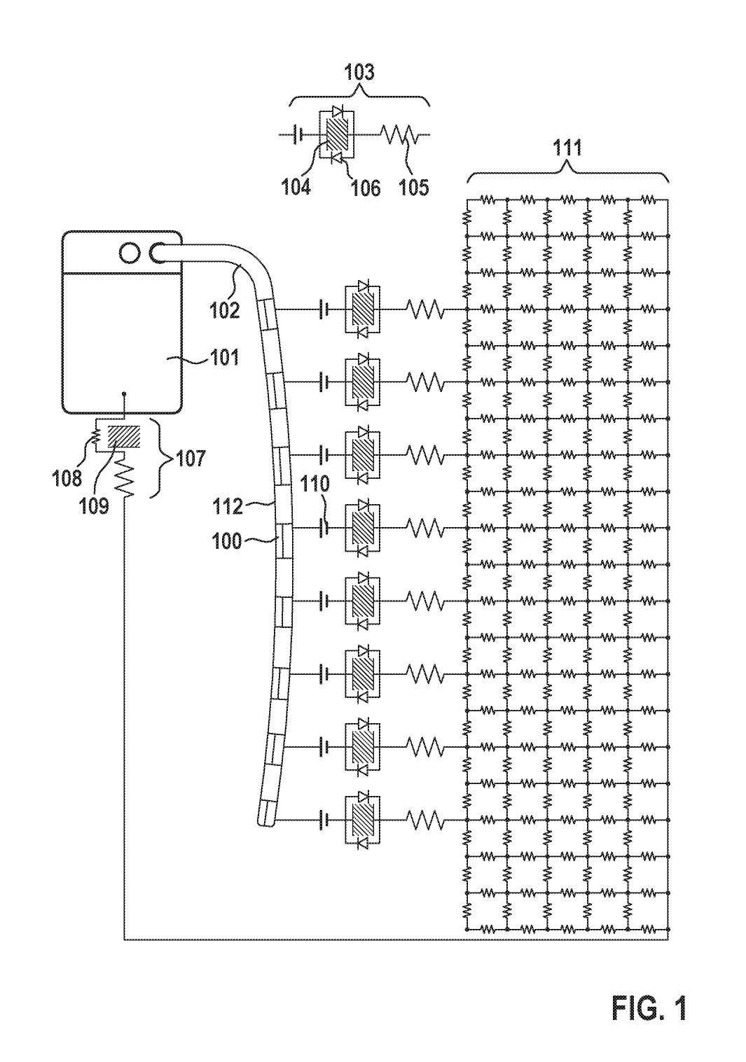

Referring now to the figures of the drawings in detail and first, particularly, to FIG. 1 thereof, there is seen a schematic representation of a classical multi-electrode system having electrodes 100 formed of an implantable pulse generator (IPG) 101 and percutaneous lead 102 for spinal cord stimulation (SCS). Although only one lead 102 and eight electrodes 100 are shown, and the embodiments are described for simplicity in terms of an SCS application, this Invention Disclosure can be extended and applied to any electrical stimulation application, implantable or not, of any number of different leads 102 and electrodes 100.

Going back to FIG. 1, each electrode-tissue interface 103 is modeled by a constant phase element 104 (also known as a fractional-pole capacitor), which models displacement currents in the electrode-tissue double layer during biphasic stimulation, in series with an access resistor 105. Diodes 106 model irreversible Faradaic reactions that may occur during biphasic stimulation depending on the charge level injected. The tissue-IPG 101 case conductive area is modeled by a similar impedance 107 where resistor 108 models leakage of the constant phase element 109. Typically, the impedance presented by the constant phase element 109 is much smaller than that of the constant phase element 104 given the much larger conductive area of the IPG 101 case.

Element 110 models the open circuit potential (OCP) of each electrode 100 measured against another conductive surface, e.g. the IPG 101 case conductive area.

For the SCS example being described, mesh 111 models the bulk tissue as a 2D array of varying resistors due to the rotational symmetry of the electrodes 100 [see Jones and Scott, “Scaling of Electrode-Tissue Interface Model Parameters In Phosphate Buffered Saline”, IEEE Transactions on Biomedical Circuits and Systems, vol. 9, issue 3, pp. 441-8, June 2015]. Percutaneous SCS lead 102 is formed of coaxial cylindrical Pt/Ir electrodes 100 separated by insulating spacers 112.

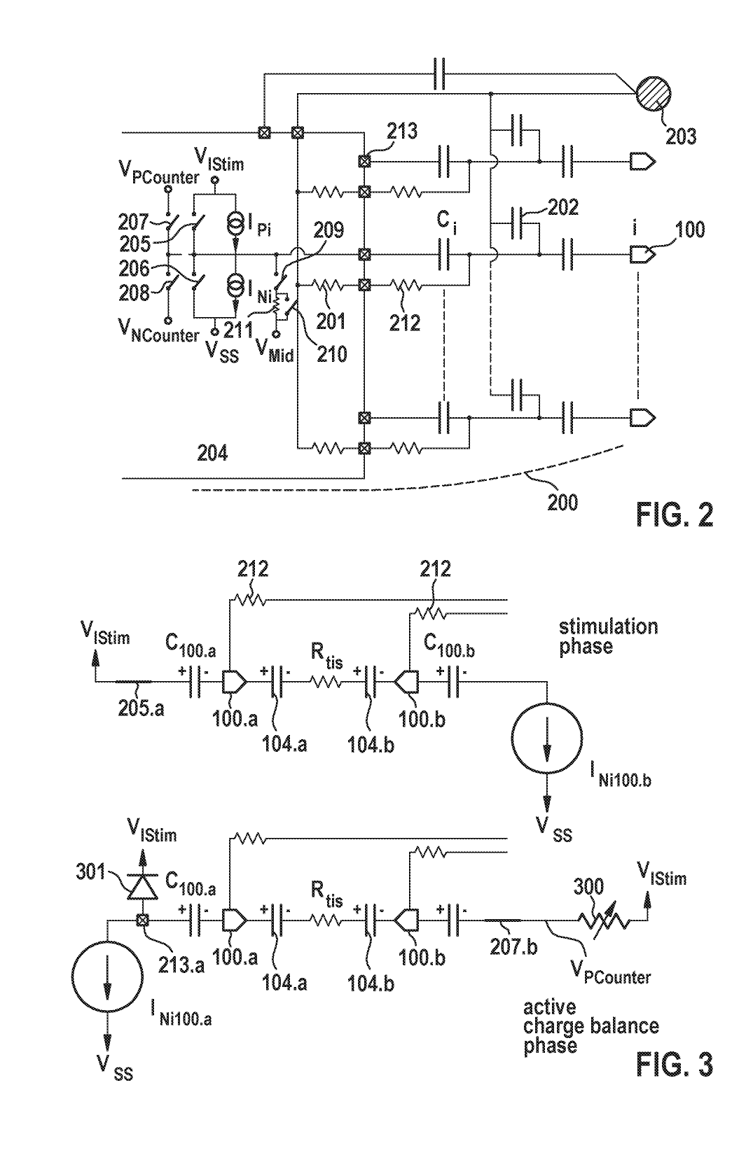

The electrodes 100 are electrically driven by the front-end 200 (in the IPG 101) shown in FIG. 2. Component Cᵢ represents the DC blocking capacitor in series with electrode i. These DC blocking capacitors Cᵢ are nominally all equal with a typical value of 10 μF±10% and with a capacitance that may reduce with accumulated biasing voltage (ceramic capacitor behavior). The “equivalent” capacitor presented by the electrode-tissue double-layer (represented by constant phase element 104), on the other hand, for a spinal cord stimulation (SCS) Pt/Ir electrode 100, has a value on the order of 4.7 μF. The present Invention Disclosure, however, assumes no particular relative values between these capacitances.

The total capacitance in series with the stimulation path is then on the order of 1.5 μF if we consider two DC blocking capacitors Cᵢ in series with two electrode-tissue double-layer capacitances modeled by element 104. Spinal cord stimulation (SCS) may require up to 12.7 μC of charge to be injected during the stimulation phase which translates into an accumulated voltage of several Vs that the active charge balance phase will see reversed and needs to handle appropriately.

Resistors 201 in FIG. 2 are bleeding resistors (hundreds of kΩ), placed in star configuration, typically utilized in IPG's front-ends 200 for passive charge neutrality. Capacitors 202, also in star configuration, provide filtering against electromagnetic interference (other protections, such as against external defibrillation pulses, are not shown). The common mode of both resistors 201 and capacitors 202 star configurations are connected to the conductive area 203 of the IPG 101 case.

An application specific integrated circuit (ASIC) 204 provides seven (7) controllable blocks for biphasic stimulation where only one may be active at any time when the respective electrode 100 is utilized for therapy delivery. Current I_{Pi} permits sourcing current through an electrode 100 from the programmable voltage V_{IStim} whereas current I_{Ni} permits sinking current to system ground V_{SS} as desired. Having sourcing and sinking currents I_{Pi} I_{Ni} independently controllable at each electrode 100 permits delivering simultaneous multi-electrode SCS therapy with active charge balancing, thus higher frequency, and applying current steering to enable targeted stimulation of specific nerve fiber populations. Analog switches 205, 206 permit connecting an electrode 100 to either V_{IStim} or V_{SS} respectively when currents of only one type are to be applied.

For an active charge balance phase, analog switches 207, 208 permit connecting the corresponding compensating voltage via terminals V_{PCounter}, V_{NCounter} respectively. Analog switches 209, 210, on the other hand, referenced to a mid-voltage V_{Mid}, and current limiting resistor 211, permit passive charge balancing. V_{Mid} may be any voltage between V_{IStim} and V_{SS} including them. Resistors 212 may be added to limit the current in the presence of externally-generated fields (e.g. defibrillation) in DC-coupled pads 213. These pads 213 may be utilized in closed-loop SCS based on evoked responses. Open-loop configurations may not require resistors 212 and associated pads 213.

Let us now describe different embodiments with different stimulation configurations. The first configuration denominated High Matching with Ohmic Voltage Drop Compensation (H-bridge equivalent), is shown in FIG. 3.

High Matching is preferably utilized with two electrodes 100.a and 100.b, and the same block or two matched blocks, i.e. current sink(s) I_{Ni} (I_{Ni100.b} and I_{Ni100.a} in this case), to deliver stimulation and actively balance the charge. During the stimulation phase, analog switch 205.a associated with electrode 100.a is connected to V_{IStim} and current sink I_{Ni100.b} associated with electrode 100.b enabled. It is worth mentioning that, although not shown in the blocks of FIG. 2, active blocks such as sink currents I_{Ni} and source currents I_{Pi} are preferably turned on through dummy load resistors and switched to tissue once the voltage across the current element is larger than the compliance voltage. This minimizes current-settling transients through tissue and allows correct compensation via ohmic voltage drop in a preferred embodiment.

During the stimulation phase of FIG. 3, DC blocking capacitors C_{100.a}, C_{100.b}, and electrode-tissue double-layer capacitances represented by elements 104.a and 104.b will charge as shown. Resistor Rₜᵢₛ models the resistive components of the electrode-tissue-electrode impedance (for example two resistors 105 with the corresponding part of 111).

For the active charge balance phase, analog switch 207.b associated with electrode 100.b is connected to V_{PCounter} and sink current I_{N100.a} associated with electrode 100.a enabled. Current sink I_{Ni100.a} may be programmed the same as I_{Ni100.b} or it may be a fraction, e.g. ½ or ¼, if asymmetric active charge balance is utilized.

As mentioned before, in a preferred embodiment, programmable resistor 300 cancels the accumulated voltage of C_{100.a}, C_{100.b} and the electrode-tissue double-layer capacitances 104.a, 104.b during the active charge balance phase to avoid turning on, for example, the parasitic/protection diode 301 connected to V_{IStim} in pad 213.a driving DC blocking capacitor C_{100.a}.

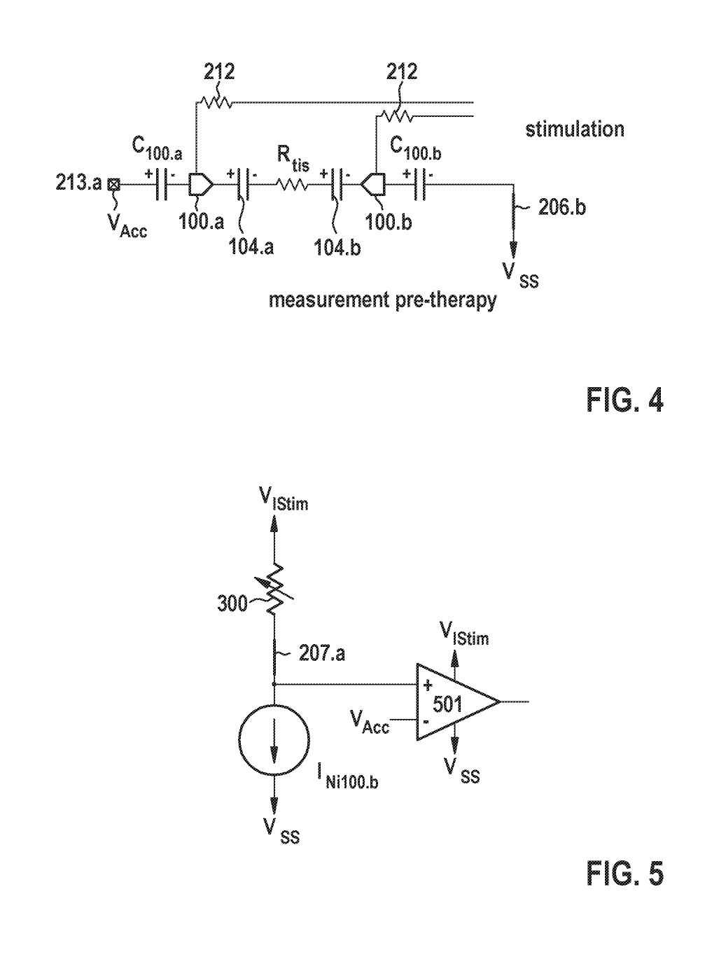

Programmable resistor 300 may be implemented via a 8-bit digital-to-analog (DAC) converter. To determine the value of 300 for a programmed therapy, a stimulation phase is first injected as shown in FIG. 3 and a measurement phase takes place after the programmed interphase period as shown in FIG. 4. Analog switch 206.b associated with electrode 100.b is closed and the accumulated voltage V_{Acc} at pad 213.a, associated with electrode 100.a, measured by ASIC 204. Passive balance is performed between electrodes 100.a and 100.b following the measurement.

According to an embodiment of the present invention, the housing of an implantable device is configured to be the return electrode.

The ASIC 204 then injects the balance current I_{Ni100.a} through the programmable resistor block 300 alone (i.e. tissue is not involved) and binary searches (via comparator 501) for the value that approximates the measured V_{Acc} as shown in FIG. 5. Analog switch 207.a associated with electrode 100.a is closed during this search.

Alternatively, there is no measurement of V_{Acc} and the programmable resistor 300 is found directly by comparison during the start of the active charge balance phase. In this alternative approach, an estimation of programmable resistor 300 to be used during the active charge balance phase is performed assuming the total capacitance in series with the stimulation path is constant (1.5 μF in the SCS example) and considering the programmed stimulation phase maximum current. During the start of the active charge balance phase, the programmable resistor 300 is quickly adjusted to an optimum value to compensate the accumulated voltage with the ohmic voltage drop.

Programmable resistor 300 has preferably a range of 20-5100 Ωin at least 256 steps.

Stimulation and active charge balance phases can then be run as shown in FIG. 3 using the High Matching configuration. Later it will be addressed how to compensate for extra accumulated voltage that may be caused by charge mismatch between the stimulation and active charge balance phases actual charges until steady-state is set or by impedance changes that may be caused by, for example, patient movement in SCS.

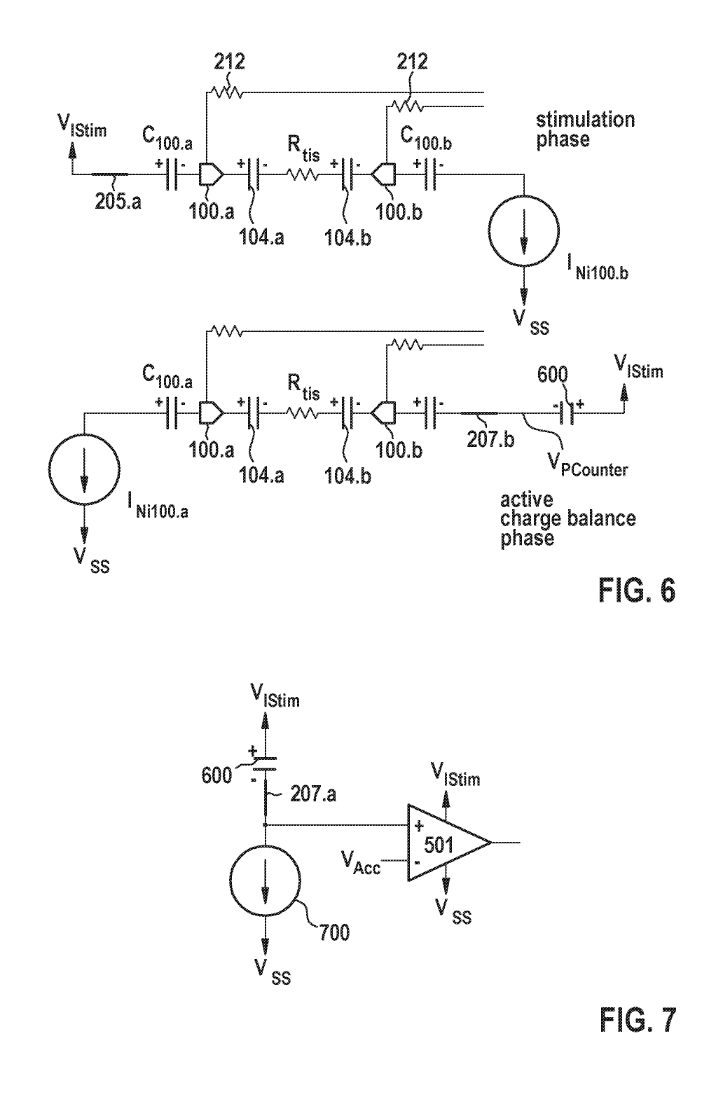

FIG. 6 shows the High Matching configuration with Capacitive Voltage Drop Compensation instead. Here, a pre-charged capacitor 600 is placed in series with the active charge balance phase as shown. Capacitor 600 is precharged to V_{Acc} and preferably has a value at least ten times larger (e.g. 15.0 μF) than the equivalent capacitance in series during the stimulation phase (typically 1.5 μF for SCS as mentioned before).

Similar to FIG. 5, capacitor 600 will be charged as shown in FIG. 7. Current 700 may be the largest programmable current in this case, e.g. 20 mA in SCS, to quickly charge capacitor 600.

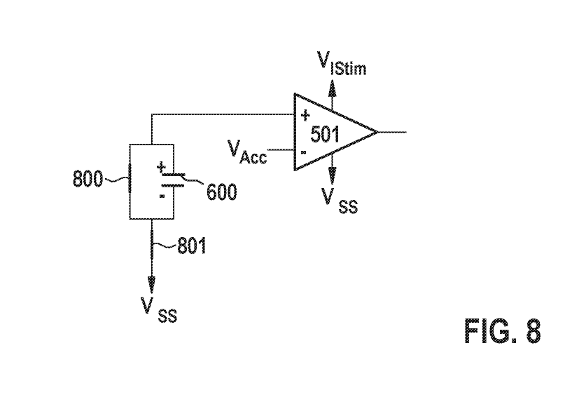

Now capacitor 600 will charge further during the active charge balance phase of FIG. 6 and the programmed V_{IStim} needs to account for. Such accumulated extra charge needs to be removed during the next stimulation phase and interphase period to bring the voltage in capacitor 600 back to V_{Acc} in preparation for the next active charge balance phase. This can be achieved with the circuit of FIG. 8. Element 800 (e.g. analog switch) passively discharges capacitor 600 and analog switch 801 permits comparing the voltage in capacitor 600 against the stored V_{Acc} to stop the discharge in capacitor 600 once its voltage reaches V_{Acc}. Alternatively, capacitor 600 is discharged directly to a voltage source V_{Acc} either passively (via elements 800, 801) or actively.

Stimulation and active charge balance phases can then be run as shown in FIG. 6 for the High Matching configuration.

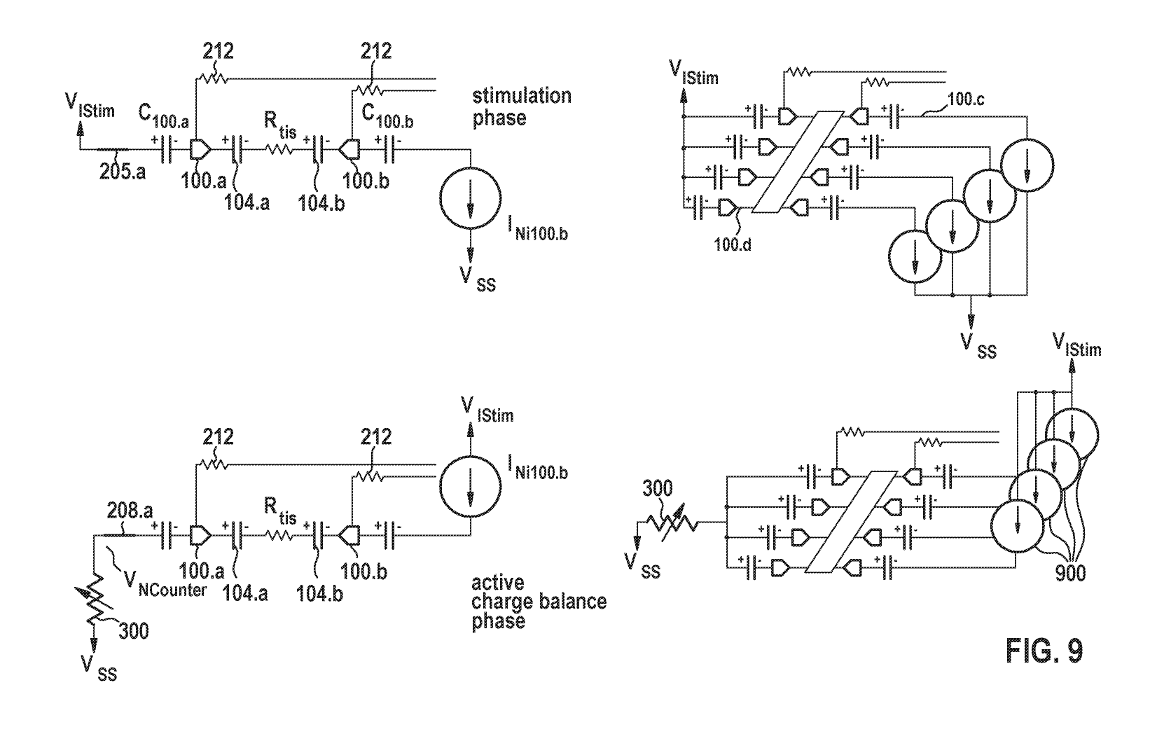

FIG. 9 shows the configuration denominated Normal Matching with Ohmic Drop Voltage Compensation. In this configuration, one or multiple currents of the same type are utilized (sinking currents I_{Ni} in the example) during the stimulation phase and the current return is either against one electrode 100.a (left side) or multiple electrodes 100.d connected to V_{IStim} via DC blocking capacitor(s) Cᵢ (right side, Z represents the complex electrode-tissue-electrode impedance).

In the active charge balance phase, currents are reversed in the electrode(s) 100.b, 100.c that were actively driven with a current during the stimulation phase, and the return electrode(s) 100.a, 100.c blocking capacitors Cᵢ connected to V_{SS} via programmable resistor 300. The value of such programmable resistor 300 may be determined in a similar way as described for the High Matching configuration with Ohmic Voltage Drop. In the case of multiple return electrodes 100.d in the stimulation phase, the accumulated voltage V_{Acc} is the maximum of the accumulated voltages, and the current to be flown in the programmable-resistor setting phase (similar to FIG. 5) the sum of active charge balancing currents 900. Alternatively, the programmable resistor 300 is pre-estimated as described before and its final value adjusted during the start of the active balance phase.

A similar compensation using capacitive voltage drop instead (as shown in FIG. 6) can be used for the Normal Matching configuration. The capacitor 600 will be connected instead of programmable resistor 300 and in the same position shown in FIG. 9.

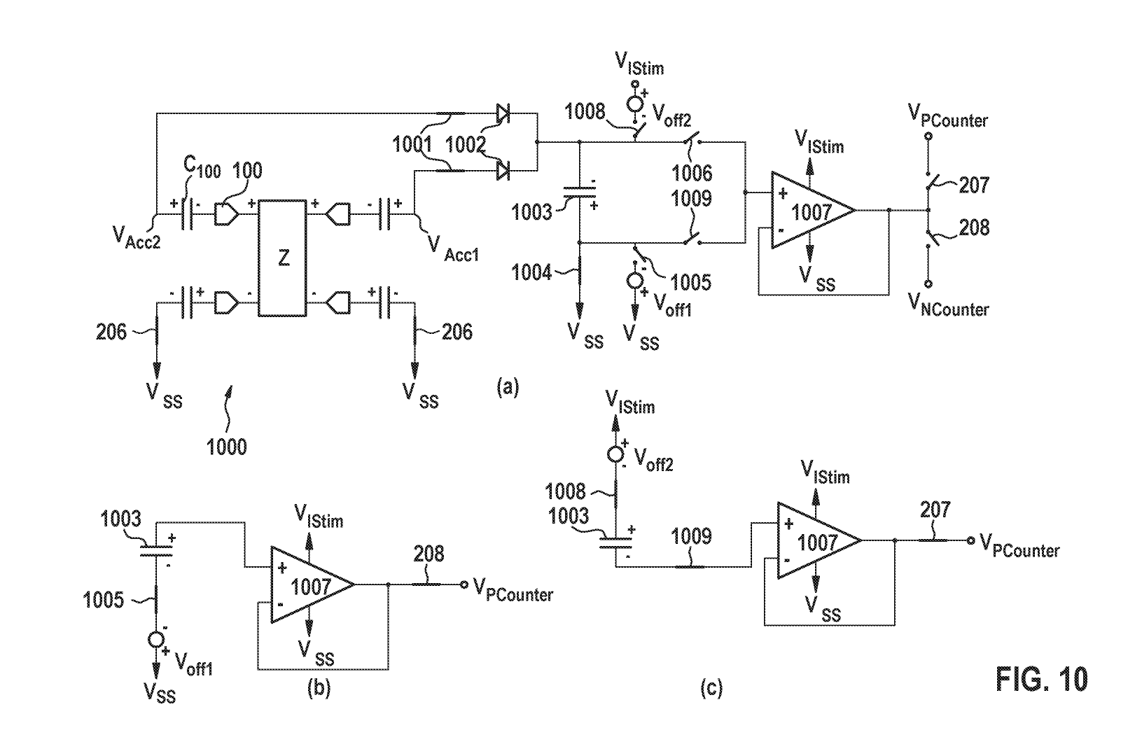

FIG. 10 describes a preferred active-element compensation embodiment using, without losing generality, a stimulation phase with four electrodes 100 that accumulate voltage as shown. During the interphase period 1000 (FIG. 10.a), analog switches 206 of the cathodic electrodes 100 during the stimulation phase are connected to system ground V_{SS} and accumulated voltages V_{ACC1} and V_{ACC2}, on the DC blocking capacitors C₁₀₀ associated with the other two electrodes 100, connected via analog switches 1001 and diodes 1002 to a capacitor 1003 connected to system ground V_{SS} via analog switch 1004. If capacitor 1003 is on the order of a few pF to tens of pF, for example, the higher V_{ACCi}(i=1, 2 in the example) is quickly sampled and held in capacitor 1003. If the active-element compensation during the active balance phase is to be performed via V_{NCounter} (see FIG. 2), analog switch 1004 is opened, and analog switches 1005, 1006 and 208 closed as shown in FIG. 10.b. Voltage V_{off1} permits adjusting voltage V_{NCounter} given the diode 1002 drop in the sampled voltage across capacitor 1003. The configuration in FIG. 10.b permits voltage follower 1007 to output a voltage V_{NCounter} equal to that held in capacitor 1003 to sink the total current during the active balance phase. Alternatively, if the active-element compensation during the active balance phase is to be performed via V_{PCounter} (see FIG. 2), analog switch 1004 is opened, and analog switches 1008, 1009 and 207 closed as shown in FIG. 10.c. Voltage V_{off2} permits adjusting voltage V_{PCounter} given the diode 1002 drop in the sampled voltage across capacitor 1003. The configuration in FIG. 10.c permits voltage follower 1007 to output a voltage V_{PCounter} equal to total overhead voltage V_{IStim} minus that held in capacitor 1003 to source the total current during the active balance phase. In a preferred embodiment, voltage follower 1007 is implemented using a class AB operation amplifier.

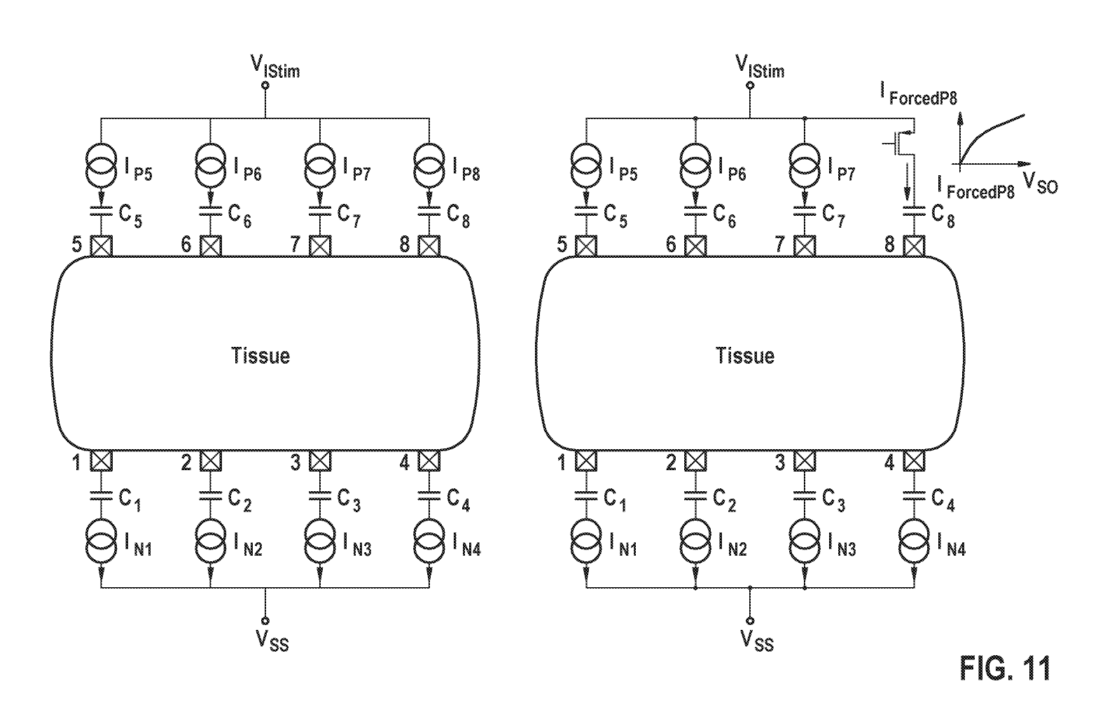

A final configuration denominated Current Steering is shown on the left side of FIG. 11 (adapted from U.S. Provisional Application No. 62/631,976). In this configuration, both sinking and sourcing currents I_{Ni}, I_{Pi} (i=1 . . . 8 in the example) are programmed to circulate through tissue during the stimulation phase having these currents different weights to “steer” the electrical field generated. Although eight electrodes 100 are shown (denominated 1 . . . 8) the configuration is not limited to this number of electrodes 100 or to having the same number of sourcing and sinking currents I_{Ni}, I_{Pi} (i=1 . . . 8 in the example).

Different implementation approaches were disclosed in U.S. Provisional Application No. 62/631,976 given the high impedance nature of current sources and the intention to avoid using an auxiliary electrode for handling the current mismatch between the sum of sinking currents I_{Ni} and the sum of sourcing currents I_{Pi} (i=1 . . . 8 in the example) during the stimulation phase. One possible implementation disclosed in U.S. Provisional Application No. 62/631,976, for the stimulation phase, is shown on the right side of FIG. 11. In such, one of the sourcing currents utilizes a transistor with lower early voltage (lower output impedance) than the others to permit adapting its current. The actual current weight of electrode 8 I_{ForcedP8} in this example will vary slightly from what it was programmed, to accommodate the mentioned mismatches, and that is acceptable as it has negligible effect on therapy.

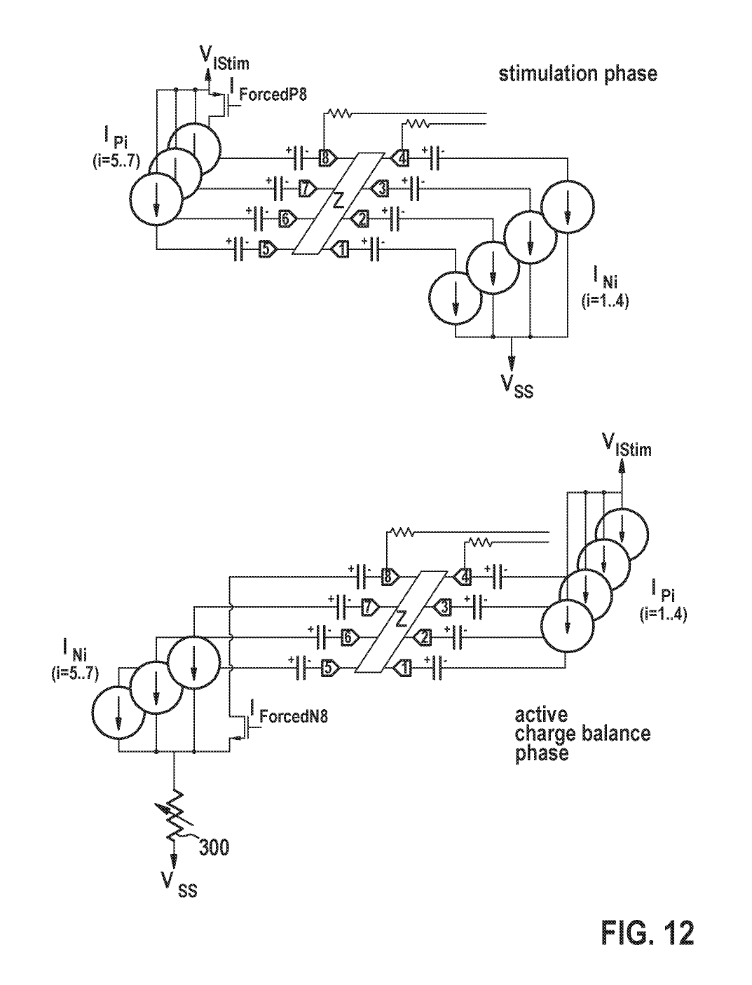

The stimulation and active charge balance phases for the Current Steering configuration are shown in FIG. 12. In the active charge balance phase, the same electrode 8 (in the example being described) is forced to drive the current mismatches between programmed sourcing currents I_{Pi} (i=1 . . . 4) and sinking currents I_{Ni} (i=5 . . . 8). Again, the value of programmable resistor 300 may be determined in a similar way as described for the High Matching configuration with Ohmic Voltage Drop (see FIG. 5). The accumulated voltage V_{Acc} is the maximum of the accumulated voltages, and the current to be flown in the programmable-resistor setting phase (similar to FIG. 5) the sum of circulating currents I_{Pi} (i=1 . . . 4).

A similar compensation using capacitive voltage drop instead (as shown in FIG. 6) can be used for the Current Steering configuration. The capacitor 600 will be connected instead of programmable resistor 300 and in the same position shown in FIG. 12. Alternatively, the Current Steering configuration can be actively-compensated via voltage follower 1007 as described in FIG. 10.

For all stimulation configurations, the present Invention Disclosure may utilize a similar determination of optimum total overhead voltage V_{IStimOpt} for efficient therapy delivery as disclosed in U.S. Provisional Application No. 62/631,976. Alternatively, V_{IStimOpt} may be pre-estimated using a measurement of complex impedances Z, in conjunction with the maximum programmed current during the stimulation phase, the maximum on resistance of either switches 205, 206, and the minimum required compliance voltage for the sinking and/or sourcing currents I_{Ni}, I_{Pi} respectively depending on the stimulation configuration. In a further alternative approach, the reactive component in the stimulation path is assumed to be fixed at 1.5 μF (for the example case of SCS) only requiring the impedance measurement to obtain the resistive components of the complex impedances Z and taking the maximum among them.

During therapy settling until steady-state is reached (i.e biphasic stimulation charge mismatch equals the charge balanced by passive balance), therapy amplitude ramp up, and during therapy delivery with long duty cycle on or continuously where the complex impedances Z may vary (e.g. caused by patient body posture changes), both the stimulation and active charge balance phases will have to be adjusted on-the-fly. For the stimulation phase, as described in U.S. Provisional Application No. 62/631,976, if any of the voltages across the current elements I_{Ni}, I_{Pi} fall below the minimum required compliance voltage to guarantee delivering the programmed current, the total voltage overhead V_{IStim} is increased in steps and later reduced to deliver the stimulation phase with the maximum efficiency.

During the active balance phase, participating pads 213 are monitored at the beginning of such phase for not exceeding a certain voltage window band (e.g. ±0.2 V, a voltage below a pn junction voltage drop) around V_{IStim} or system ground V_{SS} depending on the stimulation configuration. If any pad 213 exceeds the corresponding threshold, in the case of compensation via a programmable resistor 300, the resistor value is quickly changed to bring the pad 213 voltage within the threshold window band.

In the case of compensation via capacitor 600, if the any of the participating pads 213 exceeds on of the mentioned thresholds, capacitor 600 will be discharged as shown in FIG. 8 more or less (e.g. a fixed step of 50 mV) during the next stimulation phase and interphase period in preparation for the next active balance phase.

Finally, in the case of active-element compensation via voltage follower 1007, if the any of the participating pads 213 exceeds on of the mentioned thresholds, either voltage V_{off1} or V_{off2} can be adjusted to bring the pad 213 voltage within the threshold window band.

It will be apparent to those skilled in the art that numerous modifications and variations of the described examples and embodiments are possible in light of the above teaching. The disclosed examples and embodiments may include some or all of the features disclosed herein and the disclosed examples and embodiments are presented for purposes of illustration only. Therefore, it is the intent to cover all such modifications and alternate embodiments as may come within the true scope of this invention.

1. A medical device for electrical stimulation, the medical device comprising:
at least two electrodes;
electric circuitry for performing active charge compensation, said electric circuitry being connected to said electrodes; and
said electric circuitry being configured to perform said active charge compensation using a passive element or an active element.

2. The medical device according to claim 1, wherein said active charge compensation is performed during a stimulation phase of the medical device via an ohmic voltage drop.

3. The medical device according to claim 1, wherein said active charge compensation is performed during a stimulation phase of the medical device via a capacitive voltage drop.

4. The medical device according to claim 1, wherein said electric circuitry includes a voltage follower, and said voltage follower is configured to be used for active charge compensation.

5. The medical device according to claim 4, wherein said voltage follower is configured to emit a voltage follower output providing a total voltage overhead for said active charge compensation.

6. The medical device according to claim 1, wherein said electric circuitry includes a programmable resistor, and said programmable resistor is configured to be used for said active charge compensation.

7. The medical device according to claim 6, wherein the medical device is configured to estimate a resistance required during said active charge compensation, and said estimated resistance is programmed to said programmable resistor.

8. A method for controlling an implantable device having at least two electrodes, the method comprising the following steps:
using the electrodes of the implantable device to perform electrical stimulation; and
using a passive element or an active element to perform active charge compensation during the stimulation.

9. The method according to claim 8, which further comprises performing the active charge compensation during the stimulation via an ohmic voltage drop or a capacitive voltage drop.

10. The method according to claim 8, which further comprises performing a voltage measurement in electrical paths associated with the stimulation electrodes at least one of prior to the stimulation or at an end of an interphase period.

11. The method according to claim 10, which further comprises measuring an accumulated voltage of capacitances in the electrical paths for electrical stimulation during the voltage measurement.

12. The method according to claim 8, which further comprises:
estimating an impedance required for the active charge compensation prior to the active charge compensation; and
programming the estimated impedance to a programmable resistor.

13. The method according to claim 8, which further comprises:
estimating an impedance required for the active charge compensation during the active charge compensation; and
programming the estimated impedance to a programmable resistor.

14. The medical device according to claim 1, wherein the medical device is an implantable medical device for neurostimulation.

15. A medical device system, comprising a medical device according to claim 1.
